(19) United States
(12) Patent Application Publication
(10) Pub. No.: US 2024/0043842 A1
(43) Pub. Date: Feb. 8, 2024

(54) MUSCLE RETENTION IN AGING AND DUCHENNE MUSCULAR DYSTROPHY (DMD) THROUGH S1P INHIBITION

(71) Applicant: Washington University, St. Louis, MO (US)

(72) Inventor: Rita Brookheart, St. Louis, MO (US)

(73) Assignee: Washington University, St. Louis, MO (US)

(21) Appl. No.: 18/231,684

(22) Filed: Aug. 8, 2023

Related U.S. Application Data
(60) Provisional application No. 63/370,712, filed on Aug. 8, 2022.

(51) Int. Cl. C12N 15/113; A61K 38/46; A61K 31/7088; A61P 21/00

(52) U.S. Cl. CPC C12N 15/1137; A61K 38/465; A61K 31/7088; A61P 21/00

(57) ABSTRACT

Methods are provided of reversing muscle loss and improving mitochondrial function via site-1 protease (S1P) inhibition. Further methods are provided for treating Duchenne muscular dystrophy and age-related muscle loss via site-1 protease (S1P) inhibition.

### CROSS REFERENCE TO RELATED APPLICATIONS

This application claims the benefit of U.S. Provisional Application No. 63/370,712, filed Aug. 8, 2022, the contents of which are incorporated by reference herein in their entirety.

### STATEMENT REGARDING FEDERALLY SPONSORED RESEARCH OR DEVELOPMENT

This invention was made with government support under DK020579, DK056341, HL145326, and AR057235 awarded by the National Institutes of Health. The government has certain rights in the invention.

### INCORPORATION OF SEQUENCE LISTING XML

A computer readable form of the Sequence Listing XML containing the file named “3510075.011802 Sequence Listing.xml,” which is 22,134 bytes in size (as measured in MICROSOFT WINDOWS® EXPLORER) and generated on Aug. 3, 2023, is herein incorporated by reference. This Sequence Listing consists of SEQ ID NOs: 1-24.

### FIELD OF THE INVENTION

The present disclosure is directed to methods of reversing muscle loss and improving mitochondrial function, particularly for the treatment of diseases and conditions such as Duchenne muscular dystrophy and age-related muscle loss, via site-1 protease (S1P) inhibition.

### BACKGROUND OF THE INVENTION

Mitochondria are essential for the cellular response to physiologic and pathologic stimuli. These stimuli elicit dynamic changes in cellular energy demand and substrate availability that require cellular adaptation. Disrupted mitochondrial function can contribute to a failure in adaptation and is associated with several human diseases including muscular dystrophies and sarcopenia—skeletal muscle disorders that are associated with decreased muscle mass and mitochondrial function. Studies have focused on identifying therapeutic targets to enhance mitochondrial function and thus improve adaptability in human disease states. One key example of this in skeletal muscle is the TGF-β family of proteins that control muscle size and mitochondrial metabolic capacity. Despite advances in the understanding of the role mitochondria play in adapting to cellular stress elicited by physiologic and pathophysiologic conditions, the molecular mechanisms by which changes in mitochondrial bioenergetics are regulated and, in the case of disease, disrupted, are not yet fully understood.

Site-1 Protease (S1P; also known as subtilisin/kexin-isozyme 1 or PCSK8) coordinates the adaptive response to physiologic or pathologic stimuli through its regulated intramembrane proteolysis (RIP) of key regulators important for maintaining cellular homeostasis. S1P-mediated RIP is required for the proteolytic activation of several membrane-bound transcription factors, most notably the sterol regulatory element-binding proteins and ATF6, a key arm of the unfolded protein response. Through RIP, S1P coordinates several important signaling pathways associated with human disease and organismal development (e.g., lipid/sterol biosynthesis, lysosomal biogenesis, and the unfolded protein response). A patient was previously described with a gain-of-function, de novo mutation in S1P who exhibited altered skeletal muscle mitochondrial morphology and myoedema, but this was primarily in the context of exercise. Despite the important implications of S1P in human disease and organismal development and its potential influence on skeletal muscle, few studies have directly explored the role of S1P in muscle.

### BRIEF SUMMARY OF THE INVENTION

Various methods are disclosed herein including a method for increasing skeletal muscle mass in a subject in need thereof, the method comprising inhibiting site-1 protease in the skeletal muscle of the subject.

A further aspect of the invention is a method for treating skeletal muscle wasting in a subject in need thereof, the method comprising inhibiting site-1 protease in the skeletal muscle of the subject.

Yet another aspect is a method for improving mitochondrial function in skeletal muscle in a subject in need thereof, the method comprising inhibiting site-1 protease in the skeletal muscle of the subject.

A further aspect of the invention is a method for treating Duchenne muscular dystrophy in a subject in need thereof, the method comprising inhibiting site-1 protease in the skeletal muscle of the subject.

Other objects and features will be in part apparent and in part pointed out hereinafter.

### BRIEF DESCRIPTION OF THE SEVERAL VIEWS OF THE DRAWING

FIG. 1A depicts S1P mRNA expression levels in the indicated mouse organs. n=5 per group. Gas, gastrocnemius; TA, tibialis anterior; Sol, soleus; Hrt, heart; Liv, liver; Kid, kidney; BAT, brown adipose tissue; and WAT, white adipose tissue.

FIG. 1B depicts S1P mRNA levels in control (Wild-type) and S1P^{smKO} skeletal muscles and other organs. n=3-6 per group.

FIG. 1C depicts normalized muscle mass of soleus, gastrocnemius, and TA of 12-week-old S1P^{smKO} and WT mice. Muscle masses were normalized to body weight (BW). n=6-12 per group.

FIG. 1D depicts representative images of H&E of mid-belly sections of the gastrocnemius of S1P^{smKO} and WT mice. WT, wild type; KO, knockout.

FIG. 1E depicts representative images of fiber type staining of mid-belly sections of the gastrocnemius of S1P^{smKO} and WT mice. WT, wild type; KO, knockout.

FIG. 1F depicts fiber size (cross-sectional area) quantified from mid-belly sections of fiber-type stained images. n=4-5.

FIG. 1G depicts percent distributions of fiber types quantified from mid-belly sections of fiber-type stained images. n=4-5.

FIG. 1H depicts total number of fibers and Type IIb fiber size distribution quantified from mid-belly sections of fiber-type stained images. n=4-5.

FIG. 1I depicts normalized muscle mass of soleus, TA, and gastrocnemius of aged S1P^{smKO} and WT mice. Muscle masses were normalized to body weight (BW). n=8-12 per group.

FIG. 2A depicts pyruvate-mediated mitochondrial respiration in red gastrocnemius of S1P^{smKO} and WT mice. n=7 per group. Pyr, pyruvate; ETS, electron transport system.

FIG. 2B depicts pyruvate-mediated mitochondrial respiration in white gastrocnemius of S1P^{smKO} and WT mice. n=7 per group.

FIG. 2C depicts mitochondrial DNA content of S1P^{smKO} and WT mice normalized to 36B4. n=4-5 per group.

FIG. 2D depicts PGC1alpha and TFAM expression levels in S1P^{smKO} and WT mice normalized to 36B4. n=4-5 per group.

FIG. 2E depicts immunoblotting of oxidative phosphorylation proteins in the gastrocnemius of S1P^{smKO} and WT mice. n=4 per group.

FIG. 3A depicts a volcano plot of genes identified from RNA-Seq as significantly differentially increased and decreased in gastrocnemius of S1P^{smKO} mice relative to WT mice. MSS51 is indicated. n=4 per genotype.

FIG. 3B depicts qPCR of MSS51 mRNA expression in gastrocnemius of S1P^{smKO} and WT mice. n=5 per group.

FIG. 3C depicts qPCR of MSS51 mRNA expression in soleus of S1P^{smKO} and WT mice. n=4-7 per group.

FIG. 3D depicts knockdown efficiency of custom S1P-targeting siRNAs relative to negative control (control) siRNA in C2C12 cells by qPCR. n=3 per group.

FIG. 3E depicts qPCR analysis of MSS51 mRNA expression in C2C12 cells transiently transfected with control or S1P-targeting siRNA (S1P siRNA). n=3 per group.

FIG. 3F depicts oxygen consumption rate (OCR) of C2C12 cells transiently transfected with control or S1P siRNA plus empty vector or MSS51-Flag tagged plasmid (+MSS51) and treatment with Oligo, oligomycin; FCCP, carbonyl cyanide p-trifluoro-methoxyphenyl hydrazone; and R+AA, rotenone+antimycin A.

FIG. 3G depicts oxygen consumption rate (OCR) of C2C12 cells transiently transfected with control or S1P siRNA plus empty vector or MSS51-Flag tagged plasmid (+MSS51) and quantification of basal, maximal respiration, protein leak, ATP production, and spare respiratory capacity OCR parameters. n=5 per group of a representative experiment of two.

FIG. 4A depicts MSS51 mRNA expression of scrambled (control) and S1P siRNA transfected C2C12 cells treated with vehicle (−) or TGF-β1 (+) for 5 h after 3 days in differentiation media. n=4-5 per group.

FIG. 4B depicts a Western blot of phosphorylated Smad2 (P-Smad 2) and total Smad 2 (T-Smad 2) in scrambled (control) and S1P siRNA transfected C2C12 cells treated as in FIG. 4A. n=2-3 per group.

FIG. 4C depicts (left) a Western blot of phosphorylated AKT (P-AKT^{Ser473}) and total AKT (T-AKT) in scrambled (control) and S1P siRNA transfected C2C12 cells treated as in FIG. 4A and (right) a quantification of the Western blot shown on the left. n=2-3 per group.

FIG. 5A depicts body weight and body composition of S1P^{smKO} and WT mice. n=7-9 per group.

FIG. 5B depicts plasma TAG and cholesterol levels of S1P^{smKO} and Wild-type mice. n=7-9 per group. TAG, triacylglyceride.

FIG. 5C depicts qPCR of SREBP target gene mRNA expression levels in gastrocnemius of S1P^{smKO} and WT mice. n=5 per group.

FIG. 6A depicts Sirius Red staining of mid-belly sections of gastrocnemius of aged S1P^{smKO} and WT mice. Representative images are shown. Gastroc, gastrocnemius; TA, tibialis anterior; WT, wild type; KO, knockout.

FIG. 6B depicts qPCR analysis of collagen and fibronectin genes in the gastrocnemius of aged S1P^{smKO} and WT mice. n=4-6 per group.

FIG. 7A depicts muscle mass of 2 yr old male S1P^{smKO} and WT mice. Gastroc, gastrocnemius; TA, tibialis anterior.

FIG. 7B depicts muscle mass of 2 yr old female S1P^{smKO} and WT mice. Gastroc, gastrocnemius; TA, tibialis anterior.

Corresponding reference characters indicate corresponding parts throughout the drawings.

### DETAILED DESCRIPTION OF THE INVENTION

Here, it is shown that S1P controls mitochondrial metabolism and age-associated muscle mass loss. Although germline deletion of S1P is lethal, in the present study skeletal muscle-specific S1P knockout (S1P^{smKO}) was well tolerated and the resulting mice were overtly normal. Interestingly, glycolytic muscle fibers from S1P^{smKO} mice show increased maximal mitochondrial respiration and are resistant to age-associated muscle mass loss. The data suggest that S1P inhibits mitochondrial metabolism by controlling the mitochondrial-resident gene MSS51 and that this regulation partially occurs through the TGF-β1 signaling pathway. These data unveil a previously unknown role for S1P in the regulation of mitochondrial metabolism and muscle mass and identify a potential mechanism by which this occurs.

S1P is a key coordinator of the adaptive response to physiologic and pathologic stimuli. S1P initiates the cleavage and subsequent activation of several regulators required to maintain and restore cellular homeostasis. Much work has focused on the role of S1P in liver and bone, and its involvement in lipid/sterol homeostasis and proteostasis. To date, very little is known about the impact of S1P function on skeletal muscle and whether non-canonical functions for S1P exist in this tissue.

In the present study, the biological role of S1P in skeletal muscle was investigated using skeletal muscle-specific S1P knockout mouse line, and S1P was identified as a regulator of muscle mass and mitochondrial metabolism. Specifically, S1P^{smKO} mice have increased gastrocnemius muscle mass and, as mice age, this increase in mass is present in both gastrocnemius and soleus muscles relative to age-matched control littermates, implicating a role for S1P in age-associated muscle mass loss. S1P^{smKO} mice also have increased Complex I+Complex II respiration and elevated maximal (+FCCP) respiration. Increased maximal respiration was also recapitulated in S1P siRNA knockdown C2C12 cells. Levels of the mitochondrial-resident gene MSS51 were decreased in both SS1P^{smKO} gastrocnemius and S1P-depleted C2C12 cells. Exogenous expression of MSS51 in S1P knockdown cells obliterated the increases in maximal respiration observed, indicating S1P inhibits mitochondrial metabolism by driving MSS51 expression.

The S1P^{smKO} studies show increased mitochondrial respiration in predominately glycolytic muscle fibers, but not in oxidative fibers of the gastrocnemius. This may be due to increased abundance or activity of S1P in glycolytic fibers relative to oxidative fibers, as has been reported for the S1P substrate SREBP-1c. Moreover, MSS51 abundance is concentrated in glycolytic muscle relative to oxidative muscle. Indeed, the RNA-Seq and qPCR analysis of mouse soleus, which is primarily composed of oxidative fibers, showed no significant change in MSS51 transcript levels between S1P^{smKO} and control solei. This suggests S1P-dependent control of mitochondrial metabolism is focused on glycolytic fibers and that S1P may have an as yet unknown function in predominantly oxidative muscle types.

It was shown that MSS51 expression is decreased in the absence of S1P. In mammals, little is known about the factors that control MSS51 expression, and even less is understood about how MSS51 modulates mitochondrial respiration. Members of the TGF-β1 family of ligands (e.g., TGF-β1 and myostatin) induce MSS51 expression via an as yet unknown mechanism. Here, it was explored how S1P controls MSS51 expression, and it was shown that siRNA depletion of S1P in culture partially inhibits TGF-β1-driven MSS51 expression. One drawback to the siRNA system is that S1P was depleted, not completely deleted and thus it is possible the remaining amounts of S1P enzyme were sufficient to drive blunted, yet detectable levels of MSS51 expression. Current work is focused on determining the mechanism(s) by which S1P induces TGF-β1-driven MSS51 expression and whether this requires canonical TGF-β1 signaling pathway members including Smads.

Because depletion of S1P impacted TGF-β1-dependent MSS51 expression, this suggested a role for S1P in controlling TGF-β1 signaling pathways. To explore this possibility, it was examined whether S1P modulated TGF-β1 signaling via Smad-dependent or Smad-independent signaling pathways and demonstrated that depletion of S1P did not impact Smad 2 phosphorylation, but did increase TGF-β1-induced AKT phosphorylation. Whether S1P controls Smad activity downstream of Smad 2 phosphorylation (i.e, localization and/or activation of Smads) is not known. AKT antagonizes Smad-mediated transcription; thus the increased AKT activation in the S1P siRNA studies suggests AKT may negatively control S1P-driven MSS51 expression by inhibiting Smad activity.

Disrupted mitochondrial function and metabolism are associated with Duchenne muscular dystrophy (DMD). In mouse models of DMD (mdx mice), disrupted mitochondrial metabolism was observed early on in disease progression, suggesting disrupted mitochondrial metabolism may contribute to DMD pathophysiology. Deletion of MSS51 in mdx mice improves basal and maximal respiration relative to control mdx mice. Given the evidence that S1P promotes MSS51 expression, a role for S1P in DMD disease progression is possible.

In addition to controlling mitochondrial metabolism, TGF-β1 family ligands also control muscle mass. Increased muscle mass was observed in the gastrocnemius of 12-week-old S1P^{smKO} mice, as well as in other muscle types of aged S1P^{smKO} mice. Moreover, S1P^{smKO} mice exhibit increased fiber sizes compared to WT mice, suggestive of muscle hypertrophy. Because AKT controls cell size and AKT activation is increased in S1P-depleted cells, it is possible that the increased skeletal muscle size of the S1P^{smKO} mice may be a result of enhanced AKT activity. These data combined with the observations that S1P regulates MSS51, suggest a possible role for S1P in bridging TGF-β1-dependent control of muscle mass and mitochondrial metabolism. Since S1P inhibitors are in clinical development, inhibition of S1P as a therapeutic target to increase muscle mass in aging or other conditions associated with sarcopenia could be feasible.

In conclusion, these studies identify S1P as a regulator of mitochondrial metabolism and age-associated muscle mass loss. The data also shed light on the regulation of MSS51 by linking S1P to TGF-β1 signaling. Together, the findings uncover a previously unknown function for S1P in mitochondrial biology and implicate S1P in the adaptation to disruptions in skeletal muscle mass and metabolism.

Various methods are disclosed herein including a method for increasing skeletal muscle mass in a subject in need thereof, the method comprising inhibiting site-1 protease in the skeletal muscle of the subject.

The disclosure is further directed to a method for treating skeletal muscle wasting in a subject in need thereof, the method comprising inhibiting site-1 protease in the skeletal muscle of the subject.

Yet another aspect is a method for improving mitochondrial function in skeletal muscle in a subject in need thereof, the method comprising inhibiting site-1 protease in the skeletal muscle of the subject.

A further aspect of the invention is a method for treating Duchenne muscular dystrophy in a subject in need thereof, the method comprising inhibiting site-1 protease in the skeletal muscle of the subject.

The skeletal muscle can comprise glycolytic muscle fibers. The method can result in reduced MSS51 expression.

Inhibiting the site-1 protease in the skeletal muscle of the subject can comprise administering a genetic construct that results in lower site-1 protease protein levels in the skeletal muscle of the subject, administering a site-1 protease small molecule inhibitor to the skeletal muscle of the subject, or a combination thereof.

The genetic construct can comprise a CRISPR/Cas9 or siRNA system. These systems can include a genetic modification, such as a specific promoter, that targets inhibition of S1P specifically to skeletal muscle. This would include the diaphragm.

The administration can comprise an injection into the skeletal muscle. The administration can occur once a month, once a week, once a day, multiple times a day, or any other time frame suitable for treatment.

The subject can have a skeletal muscle wasting disease. The subject can have sarcopenia, cachexia, chronic kidney disease, a muscular dystrophy, or a combination thereof. The muscular dystrophy can be Duchenne muscular dystrophy. The cachexia can be caused by cancer, and the sarcopenia can be caused by heart failure.

The skeletal muscle can be gastrocnemius, soleus, tibialis anterior muscle, or a combination thereof.

The subject can be a mammal. The subject can be a domesticated animal or human. The subject can be a human. Domesticated animals can be pets or livestock. Specific examples of domesticated animals comprise mice, rats, dogs, cats, sheep, goats, horses, pigs, and cattle.

The subject can be geriatric. For humans, a subject is geriatric when the subject is 60 years of age or older. The subject can be greater than 60 year of age, greater than 70 years of age, greater than 80 years of age, or greater than 90 years of age.

The subject can alternatively be an adult. For humans, a subject is an adult when the subject is 18 years of age or older and under 60 years of age.

The subject can alternatively be a juvenile. For humans, a subject is a juvenile when the subject is under 18 years of age.

The subject may or may not have a site-1 protease mutation.

Having described the invention in detail, it will be apparent that modifications and variations are possible without departing from the scope of the invention defined in the appended claims.

### EXAMPLES

The following non-limiting examples are provided to further illustrate the present invention.

The mitochondrial response to changes in cellular energy demand is necessary for cellular adaptation and organ function. Many genes are essential in orchestrating this response, including the TGF-β1 target gene MSS51—an inhibitor of skeletal muscle mitochondrial metabolism. TGF-β1 signaling also controls skeletal muscle mass. Despite the implications of MSS51 in the pathophysiology of obesity and musculoskeletal disease, how MSS51 is regulated is not entirely understood. Site-1 Protease (S1P) is a key activator of several transcription factors required for cellular adaptation; however, the role of S1P in muscle and mitochondrial function are unknown. Here, S1P is identified as a negative regulator of muscle mass and mitochondrial metabolism. Disruption of S1P in mouse skeletal muscle and cultured myofibers shows S1P inhibits mitochondrial metabolism by inducing the expression of MSS51. The discovery of S1P as a regulator of mitochondrial metabolism and muscle mass expands understanding of TGF-f3 signaling and increases knowledge of cellular adaptation.

### Example 1: S1P Deletion in Skeletal Muscle Increases Muscle Mass

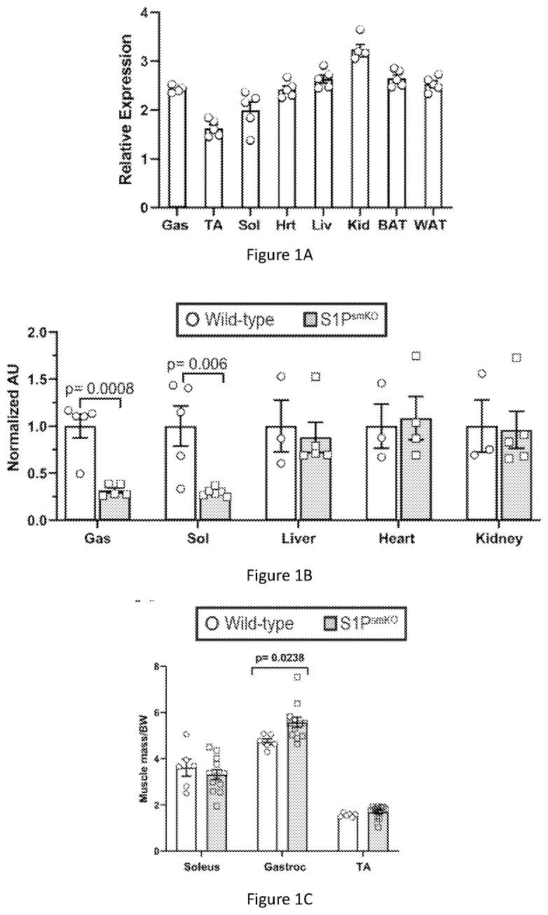

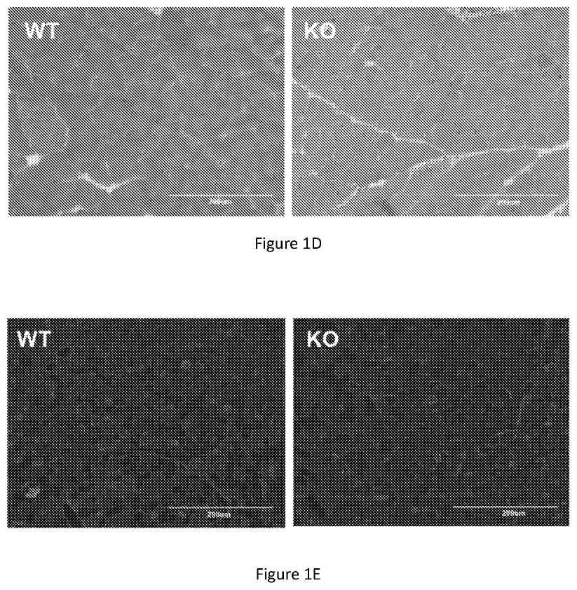

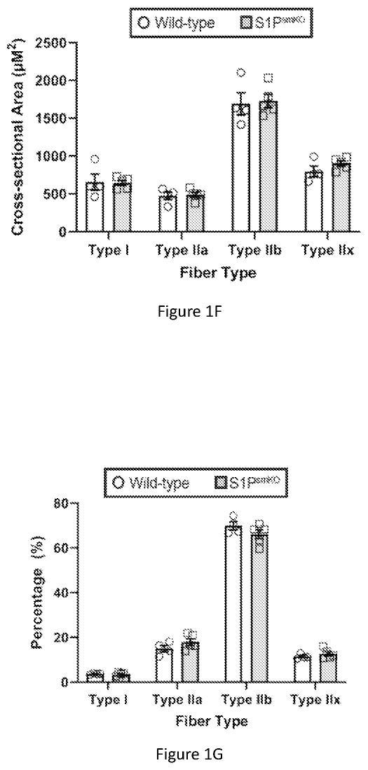

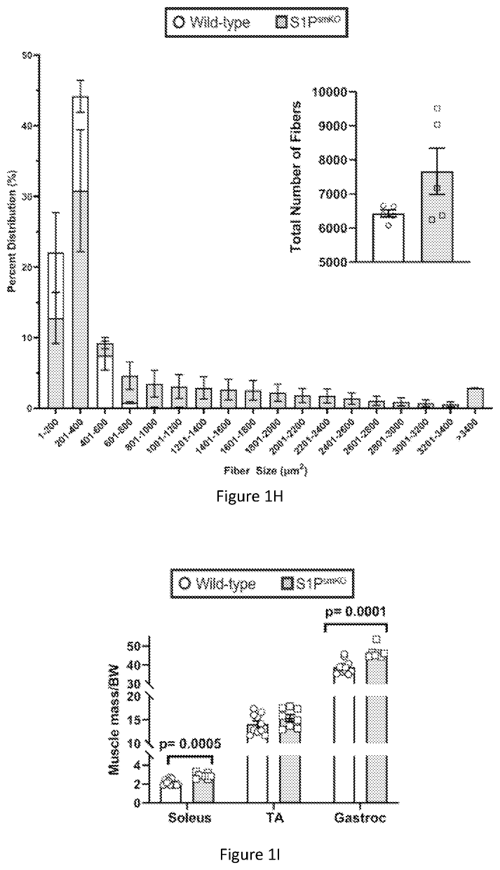

S1P function has been widely described in liver and bone, with an emphasis on its role in cellular lipid homeostasis and proteostasis. A patient with a gain-of-function mutation in S1P was recently described with a pronounced skeletal muscle phenotype (Schweitzer, G. G., et al. (2019) Mol. Genet. Genomic Med. 7, e00733). To determine the role of S1P function in skeletal muscle, S1P gene expression levels were first examined in various murine muscle groups by quantitative PCR (qPCR). S1P (encoded by Mbtps1) is expressed in mouse skeletal muscle (gastrocnemius, tibialis anterior, and soleus), with S1P mRNA levels highest in the gastrocnemius compared to other muscle groups tested (FIG. 1A). S1P gastrocnemius mRNA levels were similar to levels in the liver, an organ widely used to study S1P function (FIG. 1A).

To investigate the role of S1P in skeletal muscle, skeletal muscle-specific S1P knockout mice (S1P^{smKO}) were generated by crossing the established S1P-floxed mouse strain (Yang, J., et al. (2001) Proc. Natl. Acad. Sci. U.S.A. 98, 13607-12) with mice expressing Cre recombinase under the control of the human ACTA/promoter (FIG. 1B). Although germline deletion of S1P is embryonically lethal, homozygous S1P^{smKO} mice were viable and outwardly normal compared to floxed littermate controls (wild type, WT). Quantification of S1P mRNA in the gastrocnemius and soleus of S1P^{smKO} mice by qPCR showed a robust decrease in S1P mRNA levels compared to WT muscles (FIG. 1B).

S1P is a key regulator of SREBPs, which activate a series of target genes required for lipid and sterol biosynthesis. Deletion of S1P in mouse liver inhibits SREBP activation, decreasing plasma triglyceride and cholesterol levels, underscoring the need for S1P to maintain lipid/sterol homeostasis. Based on these observations, it was examined whether S1P^{smKO} mice had altered plasma lipid and cholesterol levels. Compared to WT littermates, S1P^{smKO} mice had normal plasma lipid and cholesterol levels, as well as normal body weight and lean and fat mass (FIGS. 5A-5B). Activation of the SREBP pathway in S1P^{smKO} was also examined by quantifying expression of SREBP target genes in skeletal muscle by qPCR, and observed no differences in target gene expression between S1P^{smKO} and WT muscles (FIG. 5C).

To examine the impact of S1P loss on skeletal muscle directly, morphological and histological analyses of S1P^{smKO} and WT muscles were performed. At 12-weeks of age, S1P^{smKO} mice exhibited a 17.6% increase in gastrocnemius mass compared to WT gastrocnemius (FIG. 1C). Soleus and tibialis anterior masses were similar between knockout and WT mice. Gross histological examination of gastrocnemius by H&E staining indicated no overt differences in fiber organization in S1P^{smKO} mice relative to WT mice (FIG. 1D). Fiber type distribution and size were examined next, and no differences in fiber size (cross-sectional area), fiber size distribution, total number of fibers, nor in the overall percentages of fiber types between knockout and WT muscle were observed (FIG. 1E-H). Together these data indicate that S1P^{smKO} mice have increased gastrocnemius mass.

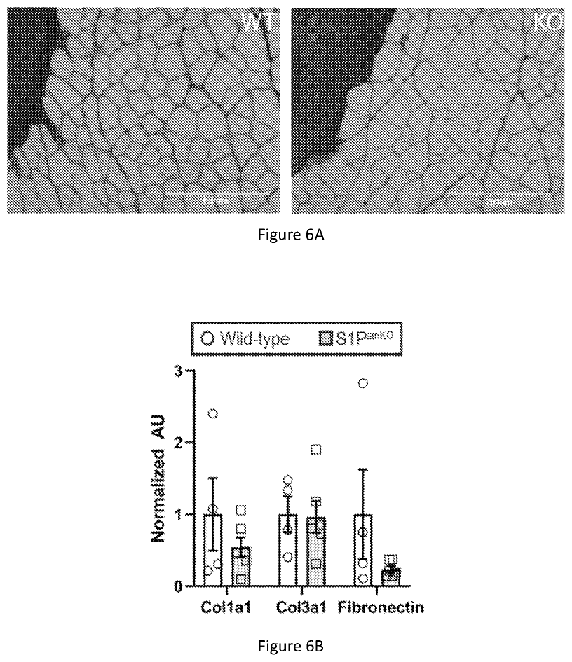

Skeletal muscle-specific deletion of S1P increased gastrocnemius muscle mass (FIG. 1C) and deletion of S1P in bone is associated with increased muscle mass in 40-week-old mice (Gorski, J. P., et al. (2016) J. Biol. Chem. 291, 4308-4322). To investigate whether S1P may control age-associated muscle mass loss, this phenotype was examined in aged (97-week-old) S1P^{smKO} and WT mice. Aged S1P^{smKO} mice had increased skeletal muscle mass in both soleus and gastrocnemius compared to age-matched WT littermates (FIG. 1I). Sirius Red staining of gastrocnemius showed no differences in collagen expansion (i.e., fibrosis) between knockout and control mice (FIG. 6A). Expression levels of fibrotic markers (Col1A1, Col3A1, and Fibronectin) were also unchanged between aged S1P^{smKO} and WT controls (FIG. 6B). These data suggest S1P negatively regulates muscle mass during aging.

### Example 2: S1P is a Negative Regulator of Mitochondrial Metabolism

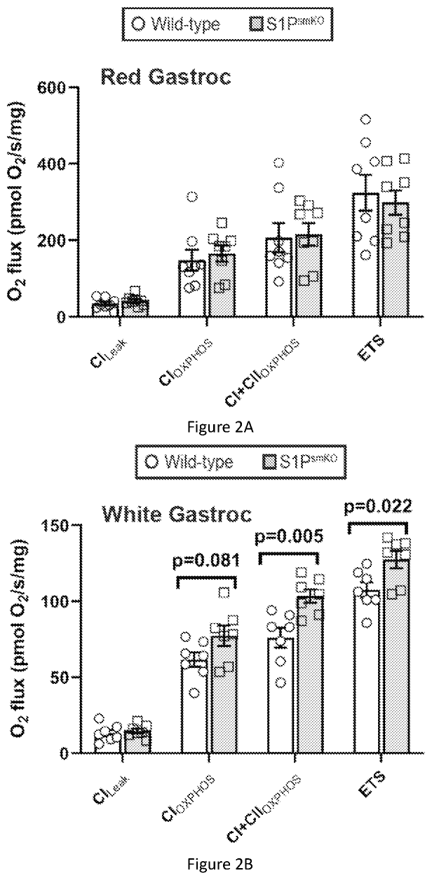

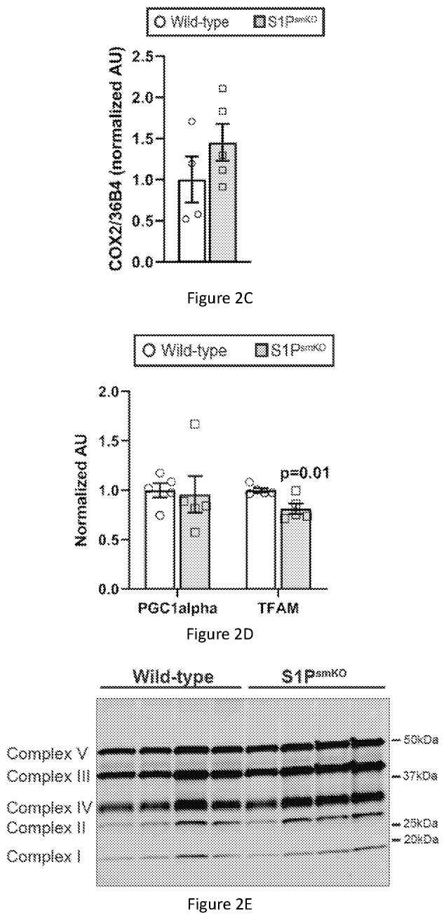

A patient with a gain-of-function mutation in S1P was previously described that exhibited altered skeletal muscle mitochondrial morphology (Schweitzer, G. G., Gan, C., Bucelli, R. C., et al. (2019) Mol. Genet. Genomic Med. 7, e00733). To examine whether skeletal muscle-specific loss of S1P impacts mitochondrial function, pyruvate-mediated mitochondrial respiration was measured in the gastrocnemius of S1P^{smKO} and WT mice. Because S1P is highly expressed in gastrocnemius and the mass of S1P^{smKO} gastrocnemius is greater than WT mice, this muscle was focused on. The gastrocnemius is composed of glycolytic (fast-twitch) and oxidative (slow-twitch) muscle fibers, which vary in their mitochondrial substrate preferences. The ‘white’ gastrocnemius, noted for its opaqueness, is primarily composed of glycolytic fibers, while the ‘red’ gastrocnemius mainly consists of oxidative fibers. Thus, the oxidative capacities of the white and red gastrocnemius were examined separately. Pyruvate-mediated mitochondrial respiration was measured in gastrocnemius fibers permeabilized with saponin. No differences were observed in mitochondrial respiration in the red gastrocnemius between S1P^{smKO} and WT mice (FIG. 2A). When mitochondrial respiration was measured in the primarily glycolytic fibers of the white gastrocnemius, Complex I+Complex II respiration and electron transport chain capacity were higher in the white gastrocnemius of S1P^{smKO} mice relative to WT mice (FIG. 2B). These findings indicate S1P is a negative regulator of mitochondrial metabolism in glycolytic muscle fibers.

To further characterize the mitochondria of S1P^{smKO} skeletal muscle, mitochondrial DNA content and expression levels of PGC-1α and TFAM were measured in S1P^{smKO} and WT gastrocnemius, markers of mitochondrial number and biogenesis. Deletion of S1P from skeletal muscle did not alter mitochondrial DNA content (FIG. 2C) and transcript levels of PGC-1α were unchanged between S1P^{smKO} and WT gastrocnemius; however, a small but significant decrease in TFAM transcript levels was observed in S1P^{smKO} muscle compared to WT controls (FIG. 2D). To determine whether changes in mitochondrial metabolism were a result of altered expression of mitochondrial electron transport chain (ETC) complexes, ETC protein levels were measured by western blot, and detected no difference in protein levels (FIG. 2E). These data suggest that the increase muscle fiber respiration was not due to increased mitochondrial abundance or altered ETC expression levels.

### Example 3: S1P Inhibits Mitochondrial Metabolism by Promoting MSS51 Expression

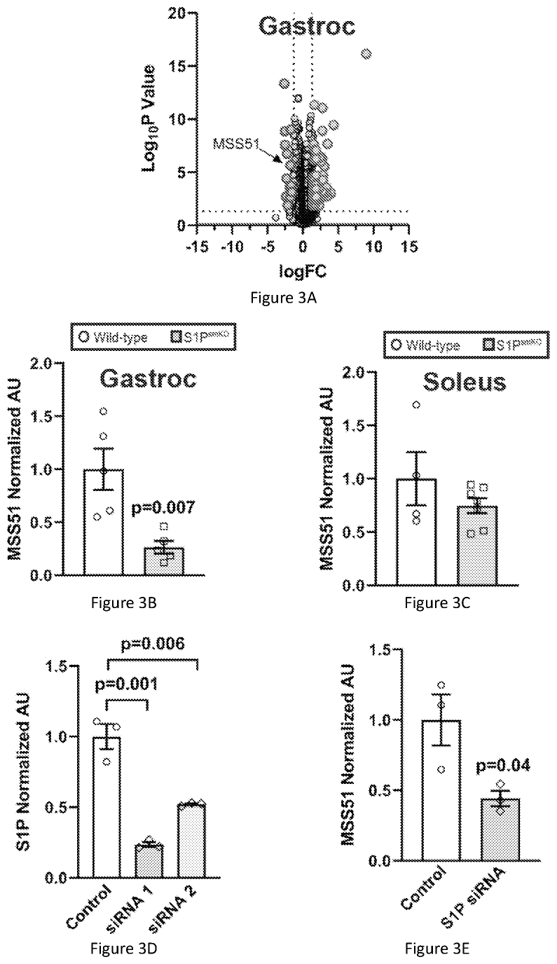

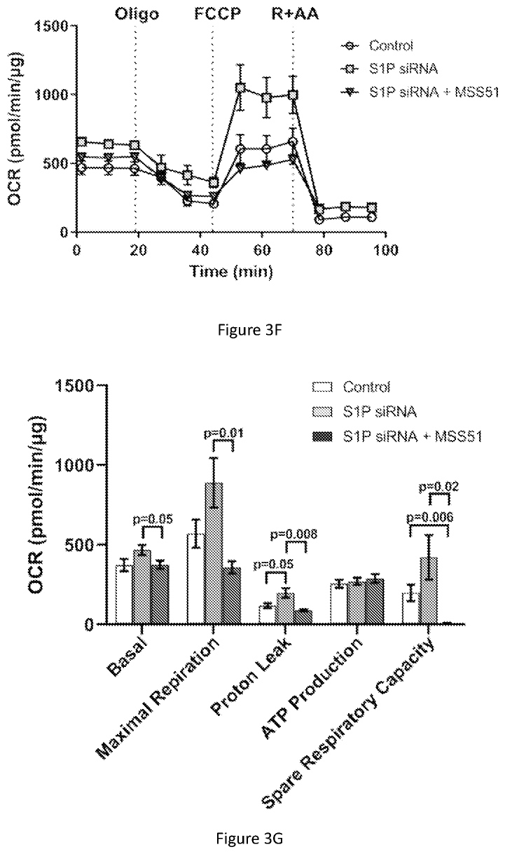

To identify the mechanism by which S1P controls mitochondrial metabolism, RNA Sequencing (RNA-Seq) was performed on RNA isolated from the gastrocnemius of 12-week-old S1P^{smKO} and WT littermates (n=4 per genotype). 75 significantly differentially expressed genes were identified; 60 up-regulated and 15 down-regulated in the gastrocnemius of S1P^{smKO} mice relative to WT mice with fold change values greater than 1.5 and p-values greater than 0.05 (FIG. 3A).

Of the significantly differentially expressed genes examined, transcript levels of the mitochondrial-resident gene MSS51 were decreased in the gastrocnemius of S1P^{smKO} mice compared to WT mice (FIG. 3A). MSS51 is primarily expressed in glycolytic muscle fibers, where it negatively regulates mitochondrial metabolism and is not involved in mitochondrial biogenesis. Indeed, the RNA-Seq analysis and subsequent qPCR analyses of MSS51 expression in the soleus (a primarily oxidative muscle), showed no change in MSS51 transcript levels in S1P^{smKO} mouse soleus relative to WT mice (FIG. 3C). These reported characteristics of MSS51 mirror the observations of S1P function in skeletal muscle.

To validate the MSS51 RNA-Seq results, MSS51 mRNA levels were measured by qPCR in the gastrocnemius of S1P^{smKO} and WT mice. The analysis showed decreased expression of MSS51 transcript levels in the gastrocnemius of S1P^{smKO} mice compared to WT mice, confirming the RNA-Seq results (FIG. 3B). To further validate the findings that loss of S1P decreases MSS51 expression, S1P was transiently knocked down in the murine C2C12 cell line using siRNA oligos to target S1P (FIG. 3D), and MSS51 transcript levels were measured by qPCR. C2C12 cells transfected with scrambled siRNA served as a negative control. Relative to scrambled siRNA cells, depletion of S1P in C2C12 cells decreased MSS51 expression, recapitulating both the RNA-Seq and in vivo qPCR results (FIG. 3E). These data indicate that S1P is a positive regulator of MSS51 expression.

Because S1P is a positive regulator of MSS51 expression and MSS51 inhibits mitochondrial metabolism, it was hypothesized that depleting S1P will reduce MSS51 expression, and thus increase mitochondrial metabolism. To test this hypothesis, Seahorse respirometry was used to quantify oxidative respiration in S1P siRNA knockdown cells that over-express either empty vector or MSS51-FLAG. Indeed, over-expressing MSS51-FLAG in S1P knockdown cells decreased basal oxygen consumption rates, maximal respiration, protein leakage, and spare respiratory capacity compared to S1P knockdown cells transfected with empty vector (FIG. 3F-G). These data show that S1P inhibits mitochondrial respiration through its control of MSS51.

### Example 4: S1P Controls MSS51 Expression through TGF-β1

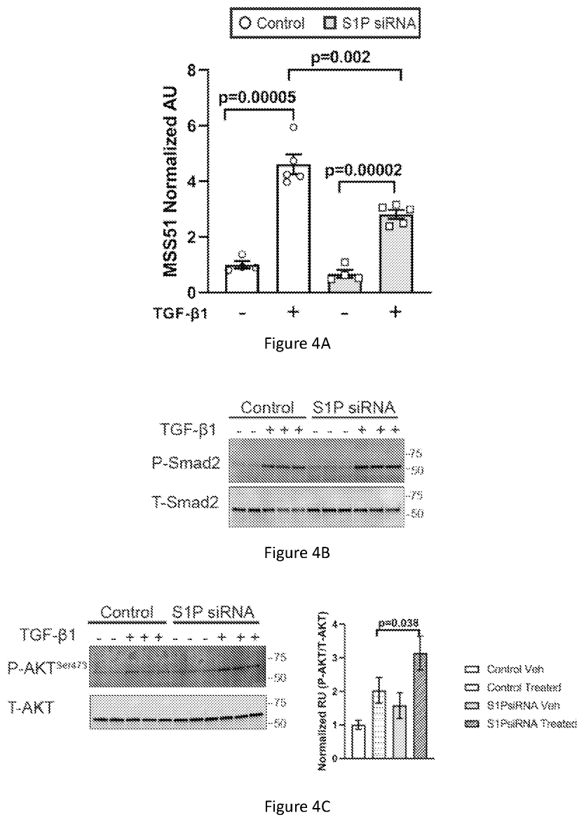

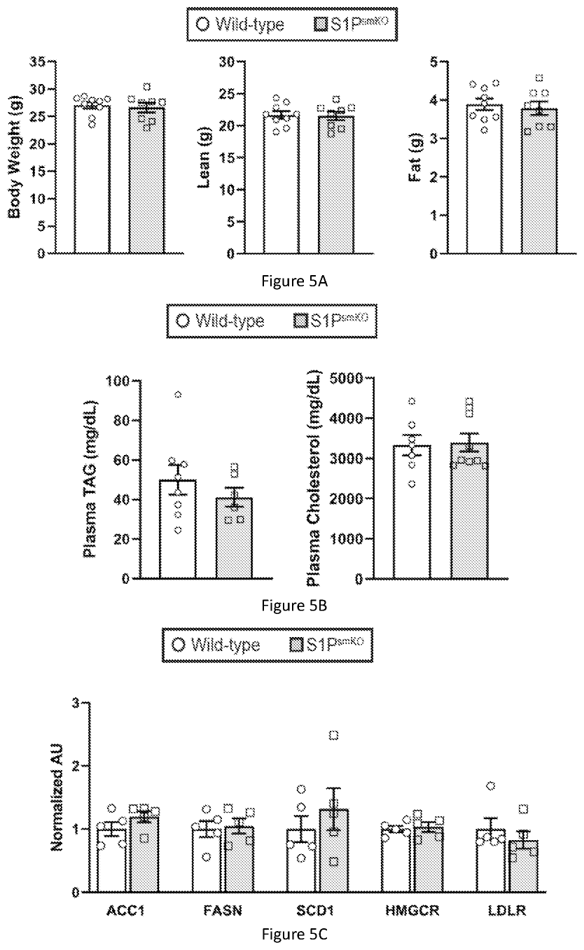

TGF-β1 and its family of ligands induce MSS51 expression through an as yet unknown mechanism. To determine if the induction of MSS51 by TGF-β1 requires S1P, S1P knockdown and scrambled (control) C2C12 cells were treated with either vehicle or recombinant TGF-β1 and measured MSS51 expression by qPCR. In the presence of TGF-β1, MSS51 expression was increased in both scrambled and S1P-depleted cells; however, MSS51 expression was significantly attenuated in S1P-depleted cells compared to scrambled control treated cells (FIG. 4A). These data demonstrate that S1P positively regulates MSS51 expression through TGF-β1.

To date, S1P has not been shown to control TGF-β1 signaling. TGF-β1 can regulate cellular function through both Smad-dependent and Smad-independent signaling pathways. To examine the function of S1P on TGF-β1 signaling and gain insight into the mechanism of S1P-driven MSS51 expression, TGF-β1-induced Smad activation was first investigated. Binding of TGF-β1 to its receptors triggers the phosphorylation and activation of the transcription factor Smad 2, thus Smad 2 phosphorylation is a positive marker of TGF-β1 Smad-dependent signaling. The phosphorylation status of Smad 2 was assessed in whole-cell lysates from vehicle (untreated) and TGF-β1-treated scrambled and S1P-depleted cells by western blotting. Smad 2 phosphorylation was not detectable in untreated cells but Smad 2 phosphorylation was equally induced in scrambled and S1P-depleted cells treated with TGF-β1 (FIG. 4B). These data suggest S1P controls TGF-β1-induced MSS51 expression independently of Smad 2 phosphorylation/activation.

TGF-β1 Smad-independent signaling was examined next, which includes TGF-131-driven AKT activation through phosphorylation of AKT on serine 473. Interestingly, TGF-131-driven AKT activation is known to antagonize Smad transcriptional activity. Levels of phosphorylated AKT^{Ser473} and total AKT were assessed in untreated and TGF-β1-treated scrambled and S1P-depleted cells. S1P-depleted cells treated with TGF-β1 had increased phosphorylated AKT compared to treated control cells (FIG. 4C). These data suggest S1P depletion leads to increased TGF-β1-dependent activation of AKT.

### Example 5: S1P Inhibition Improves Muscle Mass in Geriatric Mice

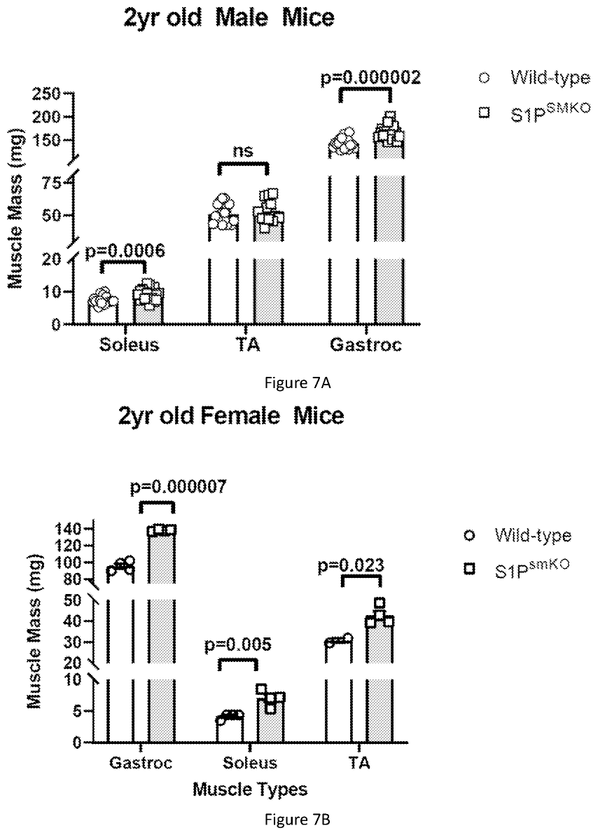

In geriatric male mice (2 yrs of age), skeletal muscle-specific deletion of S1P increased muscle mass in the gastrocnemius and soleus muscles compared to age matched control mice (FIG. 7A). In geriatric female mice (2 yrs of age), skeletal muscle-specific deletion of S1P increased muscle mass in the gastrocnemius, soleus, and tibialis anterior compared to age matched control mice (FIG. 7B). It is anticipated S1PsmKO geriatric mice will also have improved mitochondrial metabolism based on the 12 week old mouse studies presented herein. Furthermore, similar results are anticipated in Duchenne Muscular Dystrophy (DMD).

### Example 6: Methods

The following methods were used in the rest of the Examples.

### Animal Studies

All mouse studies were approved by the Institutional Animal Care and Use Committee of Washington University. Mice were maintained on a standard laboratory chow diet and group housed on a 12 h light/dark cycle. For experiments, 10-97-week-old mice were used. For blood chemistry analyses, chow-fed mice were fasted for 4 h (09:00-13:00 h) followed by tail-vein blood withdrawal. Blood was collected by venipuncture of the inferior vena cava and processed for plasma collection via centrifugation in EDTA-coated tubes, and frozen in liquid nitrogen. Skeletal muscle and other organs were harvested and either immediately snap frozen in liquid nitrogen for downstream gene expression analyses or processed for mitochondrial respiration studies or histology.

### Generation of S1P^{smKO} Mice (C57BL/6J Background)

S1P foxed mice in the C57BL/6J background were previously described (Yang, J., et al. (2001) Proc. Natl. Acad. Sci. U.S.A. 98, 13607-12) and obtained from Linda Sandell at Washington University, with generous permission from Jay Horton of University of Texas Southwestern. HSA-Cre79 mice were obtained from Jackson Laboratory (B6.Cg-Tg(ACTA1-cre)79Jme/J; Stock No. 006149) in the C57BL/6J background. S1P floxed mice were crossed with HSA-Cre79 mice to generate skeletal muscle-specific S1P knockout mice. Littermates not expressing Cre recombinase were used as controls for all experiments. Mice were genotyped for the presence of Cre recombinase and floxed S1P allele (Yang, J., et al. (2001) Proc. Natl. Acad. Sci. U.S.A. 98, 13607-12) using gene-specific primers. Primer sequences are listed in Table 1.

TABLE 1

| Gene | Forward (5′-3′) | Reverse (5′-3′) |
|---|---|---|
| S1P | ctg gct tct tgt gct ggt gg (SEQ | ctt ttc caa agc tct cgt ccc (SEQ |
| | ID NO: 1) | ID NO: 2) |
| COX2 mtDNA | ctg gtg aac tac gac tgc tag a | ggc cat aga ata acc ctg gtc (SEQ |
| | (SEQ ID NO: 3) | ID NO: 4) |
| 36B4 nDNA | acc acg aaa atc tcc aga gg | tgt cga gca ctt cag ggt ta (SEQ |
| | (SEQ ID NO: 5) | ID NO: 6) |
| 36B4 | gca gac aac gtg ggc tcc aag | ggt cct cct tgg tga aca cga agc |
| | cag at (SEQ ID NO: 7) | cc (SEQ ID NO: 8) |
| MSS51 | agg tct gtc cca gtt gat cct | att gga aag gcc atg agg gag |
| | (SEQ ID NO: 9) | (SEQ ID NO: 10) |
| TFAM | agg ctt gga aaa atc tgt ctc | tgc tct tcc caa gac ttc att (SEQ |
| | (SEQ ID NO: 11) | ID NO: 12) |
| PGC-1alpha | aga caa atg tgc ttc caa aaa gaa | gaa gag ata aag ttg ttg gtt tgc c |
| | (SEQ ID NO: 13) | (SEQ ID NO: 14) |
| ACC1 | atg ggc gga atg gtc tct ttc | tgg gga cct tgt ctt cat cat (SEQ |
| | (SEQ ID NO: 15) | ID NO: 16) |
| FASN | gtc tgg aaa gct gaa gga tct c | tgc ctc tga acc act cac ac (SEQ |
| | (SEQ ID NO: 17) | ID NO: 18) |
| SCD1 | ttc ttg cga tac act ctg gtg c | cgg gat tga atg ttc ttg tcg t (SEQ |
| | (SEQ ID NO: 19) | ID NO: 20) |
| HMGCR | cca cgc agc aaa cat tgt ca | gca ggc ttg ctg agg tag aa (SEQ |
| | (SEQ ID NO: 21) | ID NO: 22) |
| LDLR | acc tgc cga cct gat gaa ttc | gca gtc atg ttc acg gtc aca (SEQ |
| | (SEQ ID NO: 23) | ID NO: 24) |

### Body Composition

ECHO MRI was used to measure body composition in unanesthetized mice using an ECHOMRI 3-1 (ECHO Medical Systems).

### Serum Metabolites

Plasma triglyceride and cholesterol levels were measured enzymatically via the Infinity triglyceride (TR22421) and cholesterol (TR13421) assay kits (Thermo Fisher) as per manufacturer's instructions.

### Histological Analyses

Tragacanth gum was placed on top of corks and fresh muscles were vertically placed in the gum so that ¼ of the muscle was embedded. Samples were submerged in cold (−150° C.) isopentane as described (Guardiola, O., et al. (2017) Vis. Exp. 10.3791/54515) for 20 s, and immediately stored at −80° C. until sectioning. Frozen muscles were transversely cryosectioned into 10 μM thick sections at the mid-belly on a cryostat (Leica Biosystems). Sections were stained with haematoxylin (H&E), Sirius Red or immunostained against myosin heavy chain isoforms (type I (BA-F8), type IIa (SC-71), type IIx, and type IIb (BF-F3); Developmental Studies Hybridoma Bank) and laminin (ab11575, Abcam). Cross sectional area, fiber size, and fiber type distribution of each fiber type was quantified from immunostained fiber type images as reported previously (Biltz, N. K., et al. (2020) J. Physiol. 598, 2669-2683).

### Gene Expression Analysis

Total RNA was isolated from C2C12 cells, skeletal muscle, heart, adipose, kidney, and liver with RNA STAT-60 (Tel-Test Inc) as per manufacturer's instructions. For tissues, RNA was isolated by disrupting tissue in RNA STAT-60 using 5 mM steel beads (Qiagen) and a TissueLyser II (Qiagen). RNA was reverse transcribed into cDNA using the High-Capacity cDNA Reverse Transcription Kit (Applied Biosystems). Quantitative real-time PCR was performed using Power SYBR green (Applied Biosystems) and transcripts quantified on an ABI QuantiStudio 3 sequence detection system (Applied Biosystems). Data was normalized to 36B4 expression, unless otherwise noted, and results analyzed using the 2^{-ΔΔCt} method and reported as relative units to controls. Primer sequences are listed in Table 1.

### RNASeq Analysis

Gastrocnemius and soleus were harvested from 12-week-old male floxed (wild type) and S1P skeletal muscle-specific knockout mice and immediately snap frozen in liquid nitrogen for a total of n=4 mice per genotype examined. RNA was isolated from tissue as described above. RNA was DNase I treated as per manufacturer's instructions (RNase-Free DNase Set, Qiagen) then cleaned up and eluted with RNAse and DNAse free molecular grade water (RNeasy MinElute Cleanup Kit, Qiagen), followed by quantification (NanoDrop, ThermoFischer Scientific). RNA with RIN values greater than 8 were accepted for RNASeq. Samples were prepared according to library kit manufacturer's protocol, indexed, pooled, and sequenced on an Illumina HiSeq. Basecalls and demultiplexing were performed with Illumina's bcl2fastq software and a custom python demultiplexing program with a maximum of one mismatch in the indexing read. RNA-seq reads were aligned to the Ensembl release 76 primary assembly with STAR version 2.5.1a (Dobin, A., et al. (2013) Bioinformatics. 29, 15-21). Gene counts were derived from the number of uniquely aligned unambiguous reads by Subread:featureCount version 1.4.6-p5 (Liao, Y., et al. (2014) Bioinformatics. 30, 923-930). Isoform expression of known Ensembl transcripts were estimated with Salmon version 0.8.2 (Patro, R., et al. (2017) Nat. Methods. 14, 417-419). Sequencing performance was assessed for the total number of aligned reads, total number of uniquely aligned reads, and features detected. The ribosomal fraction, known junction saturation, and read distribution over known gene models were quantified with RSeQC version 2.6.2 (Wang, L., et al. (2012) Bioinformatics. 28, 2184-2185).

All gene counts were then imported into the R/Bioconductor package EdgeR (Robinson, M. D., et al. (2010) Bioinformatics. 26, 139-140) and TMM normalization size factors were calculated to adjust samples for differences in library size. Ribosomal genes and genes not expressed in the smallest group size minus one sample greater than one count-per-million were excluded from further analysis. The TMM size factors and the matrix of counts were then imported into the R/Bioconductor package Limma (Ritchie, M. E., et al. (2015) Nucleic Acids Res. 43, e47). Weighted likelihood based on the observed mean-variance relationship of every gene and sample were then calculated for all samples with the voomWithQualityWeights (Liu, R., et al. (2015) Nucleic Acids Res. 10. 1093/NAR/GKV412). The performance of all genes was assessed with plots of the residual standard deviation of every gene to their average log-count with a robustly fitted trend line of the residuals. Differential expression analysis was then performed to analyze differences between conditions and the results were filtered for only those genes with Benjamini-Hochberg false-discovery rate adjusted p-values less than or equal to 0.05.

The accession number for the RNA-seq data reported in this study is deposited at NCBI GEO under accession number GSE199014 located at https://www.ncbi.nlm.nih.gov/geo/query/acc.cgi?acc=GSE199014.

### Cell Culture

All cells were grown at 37° C. with 5% CO₂. C2C12 cells (ATCC) were grown in DMEM supplemented with 10% fetal bovine serum and 1% pen-strep. To differentiate C2C12 myoblasts into myotubes, cells were grown to 80% confluency, washed with 1×PBS and grown in DMEM supplemented with 2% horse serum for 2-3 days as indicated in the methods.

### siRNA Studies

C2C12 cells were plated onto 6-well plates at a 2×10⁵ density and 24 h later transfected with either negative control siRNA (Negative Control No. 1 siRNA, Life Technologies) or custom siRNAs targeting S1P (Silencer Select siRNAs, Life Technologies) using Lipofectamine RNAiMAX as per manufacturer's instructions. After 48 h, cells were either harvested for gene expression analysis or differentiated with DMEM supplemented with 2% horse serum. Two days after differentiation, cells were harvested for gene expression analysis. For TGF-β1 treatment studies, three days post-differentiation, cells were treated with 50 ng/ml TGF-β1 (R&D) for 5 h then harvested for downstream endpoints.

### Seahorse OCR Analysis

Cellular respiration was measured on a Seahorse XFe24 Analyzer (Agilent). C2C12 cells were plated onto 24-well Seahorse XF24 cell culture microplates at a 8,000 cell density and 24 h later co-transfected with either negative control siRNA (Negative Control No. 1 siRNA, Life Technologies) with empty vector (pCMV6-Entry; Origene PS100001) or a custom siRNA targeting S1P (Silencer Select siRNAs, Life Technologies) with empty vector (pCMV6-Entry; Origene PS100001) or mouse MSS51-Myc-FLAG (mouse cDNA clone; Origene MR217897) using Lipofectamine 2000 as per manufacturer's instructions. After 48 h, cells were washed with 1×PBS and switched to differentiation media (DMEM and 2% horse serum) for 3 days. Cells were fed Seahorse XF-DMEM with 1 mM pyruvate, 10 mM glucose, and 2 mM glutamine and incubated in a CO²-free incubator at 37° C. for 1 h. Basal oxygen consumption rates were measured first followed by OCR measurements upon sequential addition of oligomycin (1 μM), carbonyl cyanide p-trifluoro-methoxyphenyl hydrazone (FCCP; 1 μM), and rotenone and actinomycin A (0.5 μM each) as per the Seahorse Mitochondrial Stress Test protocol. After completion of the assay, whole protein lysates for each well were quantified by BCA assay and total protein amounts were used for normalization of Seahorse data using Wave Software (Agilent). Basal OCR, maximal respiration, protein leakage, and spare respirometry capacity were calculated using the Seahorse XF Cell Mito Stress Test Report Generator via Wave Software (Agilent) normalized to total protein levels.

### Preparation of Permeabilized Muscle Fibers and High-Resolution Respirometry

Freshly isolated red and white gastrocnemius sections were immersed in cold BIOPS (10 mM EGTA, 50 mM MES, 0.5 mM DTT, 6.56 mM MgCL2, 5.77 mM ATP, 20 mM Imidazole and 15 mM phosphocreatine, pH 7.1). Tissue was trimmed of surrounding fat tissue and fibers mechanically separated on ice. Separated fibers were permeabilized with BIOPS solution containing 50 μg/mL saponin for 20 minutes at 4° C. Following permeabilization, fibers were washed for 15 minutes in ice cold mitochondrial respiration solution (MIR05, 0.5 mM EGTA, 3 mM Mg2, 60 mM K-lactobionate, 20 mM taurin, 10 mM KH2PO4, 20 mM HEPES, 110 mM sucrose and 1 g/L BSA, pH 7.1). Fibers were then blotted dry, weighed (3-5 mg total tissue weight) and placed in a Oxygraph-2K (OROBOROS Instruments) chamber containing 2 mL of 37° C. MirO5 (supplemented with 10 μM blebbistatin and 20 mM creatine). Routine oxygen consumption was measured by the sequential addition of the following substrates: malate (0.5 mM), glutamate (10 mM) and pyruvate (5 mM) to assess complex I mediated LEAK respiration. Adenosine diphosphate (ADP, 5 mM) to assess maximal complex I maximal respiration followed by succinate (10 mM) to measure OXPHOS (complex I and II mediated respiration). The uncoupling agent FCCP (carbonyl cyanide p-trifluoro-methoxyphenyl hydrazone, 0.5 μM, titrated 3×) was then added to determine maximal electron transport system (ETS) capacity. A period of stabilization followed the addition of each substrate and the oxygen flux per mass was recorded using the DatLab Software (OROBOROS Instruments).

### Mitochondrial Content

DNA was isolated from 25 mg of either whole or white gastrocnemius of S1P^{smKO} and WT mice using the DNeasy Blood & Tissue Kit (Qiagen) following manufacturer's instructions. DNA concentrations were measured via NanoDrop (Thermo Scientific) and 10 ng of DNA was used for qPCR analysis using primers specific to a mitochondrial encoded gene DNA (Cox2) and nuclear encoded gene (36B4) and Power SYBR Green (Applied Biosystems). Transcripts were quantified on an ABI QuantiStudio 3 sequence detection system (Applied Biosystems) and Cox2 expression was normalized to 36B4 expression, and results analyzed using the 2^{-ΔΔCt} method and reported as relative units to controls. Primer sequences are listed in Table 1.

### Western Blotting

Skeletal muscle whole protein lysates were generated by homogenizing tissues in lysis buffer (20 mM Tris, 15 mM NaCl, 1 mM EDTA, 0.2% NP-40, and 10% glycerol) supplemented with 2× Protease Complete cocktail tablet (Roche) and 1× Phosphatase Inhibitors (Roche, Mannheim, Germany) with stainless steel beads in a TissueLyzer II (Qiagen). Protein lysates were rotated for 45 min at 4° C., followed by centrifugation at 15,000×g for 15 min at 4° C. Protein was quantified by bicinchoninic acid assay (BCA, Pierce Biotechnology), equal amounts of protein were resolved on a 4-15 SDS-PAGE gradient gel (Bio-Rad), and transferred to PVDF-FL membrane (MilliporeSigma). Blots were probed with appropriate primary and secondary antibodies and proteins visualized by LI-COR Odyssey imaging system. To visualize phosphorylated Smad 2, blots were incubated with SignalFire ECL Reagent (Cell Signaling) and protein visualized with a BioRad ChemiDoc XRS+. Primary antibodies used: OXPHOS (MS604-300, Abcam); Phospho-Smad2 (Ser465/467) (3108, Cell Signaling); Smad 2 (5339, Cell Signaling); Phospho-AKT (4060, Cell Signaling), and Total-AKT (2920, Cell Signaling). Densitometry analysis was performed using LI-COR Image Studio Lite.

### Statistical Analysis

Data were analyzed using either Excel or GraphPad Prism version 9, unless indicated otherwise in Methods. A p-value<0.05 was considered statistically significant. Data are reported as ±SEM. Unpaired two-tailed Student's t-tests were used.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

1. A method for increasing skeletal muscle mass in a subject in need thereof, the method comprising inhibiting site-1 protease in the skeletal muscle of the subject.

2. A method for treating skeletal muscle wasting in a subject in need thereof, the method comprising inhibiting site-1 protease in the skeletal muscle of the subject.

3. A method for improving mitochondrial function in skeletal muscle in a subject in need thereof, the method comprising inhibiting site-1 protease in the skeletal muscle of the subject.

4. The method of claim 3, wherein the skeletal muscle comprises glycolytic muscle fibers.

5. The method of claim 4, wherein the method results in reduced MSS51 expression.

6. The method of claim 5, wherein inhibiting the site-1 protease in the skeletal muscle of the subject comprises administering a genetic construct that results in lower site-1 protease protein levels in the skeletal muscle of the subject, administering a site-1 protease small molecule inhibitor to the skeletal muscle of the subject, or a combination thereof.

7. The method of claim 6, wherein the genetic construct comprises a CRISPR/Cas9 or siRNA system.

8. The method of claim 1, wherein the administration comprises an injection into the skeletal muscle.

9. The method of claim 1, wherein the administration occurs once a month, once a week, once a day, or multiple times a day.

10. The method of claim 1, wherein the subject is geriatric.

11. The method of claim 3, wherein the subject has a skeletal muscle wasting disease.

12. The method of claim 11, wherein the subject has sarcopenia, cachexia, chronic kidney disease, a muscular dystrophy, or a combination thereof.

13. The method of claim 12, wherein the muscular dystrophy is Duchenne muscular dystrophy.

14. The method of claim 12, wherein the cachexia is caused by cancer.

15. The method of claim 12, wherein the sarcopenia is caused by heart failure.

16. The method of claim 3, wherein the skeletal muscle is gastrocnemius, soleus, tibialis anterior muscle, or a combination thereof.

17. The method of claim 3, wherein the subject is a mammal.

18. The method of claim 17, wherein the subject is a domesticated animal or human.

19. The method of claim 18, wherein the subject is a human.

20. The method of claim 19, wherein the subject is greater than 60 years of age, greater than 70 years of age, greater than 80 years of age, or greater than 90 years of age.

21. The method of claim 3, wherein the subject does not have a site-1 protease mutation.


SEQUENCE LISTING (24 sequences)

SEQ ID NO: 1 - DNA, 20 bp, synthetic construct
  source 1..20: mol_type = other DNA
ctggcttcttgtgctggtgg

SEQ ID NO: 2 - DNA, 21 bp, synthetic construct
  source 1..21: mol_type = other DNA
cttttccaaagctctcgtccc

SEQ ID NO: 3 - DNA, 22 bp, synthetic construct
  source 1..22: mol_type = other DNA
ctggtgaactacgactgctaga

SEQ ID NO: 4 - DNA, 21 bp, synthetic construct
  source 1..21: mol_type = other DNA
ggccatagaataaccctggtc

SEQ ID NO: 5 - DNA, 20 bp, synthetic construct
  source 1..20: mol_type = other DNA
accacgaaaatctccagagg

SEQ ID NO: 6 - DNA, 20 bp, synthetic construct
  source 1..20: mol_type = other DNA
tgtcgagcacttcagggtta

SEQ ID NO: 7 - DNA, 26 bp, synthetic construct
  source 1..26: mol_type = other DNA
gcagacaacgtgggctccaagcagat

SEQ ID NO: 8 - DNA, 26 bp, synthetic construct
  source 1..26: mol_type = other DNA
ggtcctccttggtgaacacgaagccc

SEQ ID NO: 9 - DNA, 21 bp, synthetic construct
  source 1..21: mol_type = other DNA
aggtctgtcccagttgatcct

SEQ ID NO: 10 - DNA, 21 bp, synthetic construct
  source 1..21: mol_type = other DNA
attggaaaggccatgagggag

SEQ ID NO: 11 - DNA, 21 bp, synthetic construct
  source 1..21: mol_type = other DNA
aggcttggaaaaatctgtctc

SEQ ID NO: 12 - DNA, 21 bp, synthetic construct
  source 1..21: mol_type = other DNA
tgctcttcccaagacttcatt

SEQ ID NO: 13 - DNA, 24 bp, synthetic construct
  source 1..24: mol_type = other DNA
agacaaatgtgcttccaaaaagaa

SEQ ID NO: 14 - DNA, 25 bp, synthetic construct
  source 1..25: mol_type = other DNA
gaagagataaagttgttggtttgcc

SEQ ID NO: 15 - DNA, 21 bp, synthetic construct
  source 1..21: mol_type = other DNA
atgggcggaatggtctctttc

SEQ ID NO: 16 - DNA, 21 bp, synthetic construct
  source 1..21: mol_type = other DNA
tggggaccttgtcttcatcat

SEQ ID NO: 17 - DNA, 22 bp, synthetic construct
  source 1..22: mol_type = other DNA
gtctggaaagctgaaggatctc

SEQ ID NO: 18 - DNA, 20 bp, synthetic construct
  source 1..20: mol_type = other DNA
tgcctctgaaccactcacac

SEQ ID NO: 19 - DNA, 22 bp, synthetic construct
  source 1..22: mol_type = other DNA
ttcttgcgatacactctggtgc

SEQ ID NO: 20 - DNA, 22 bp, synthetic construct
  source 1..22: mol_type = other DNA
cgggattgaatgttcttgtcgt

SEQ ID NO: 21 - DNA, 20 bp, synthetic construct
  source 1..20: mol_type = other DNA
ccacgcagcaaacattgtca

SEQ ID NO: 22 - DNA, 20 bp, synthetic construct
  source 1..20: mol_type = other DNA
gcaggcttgctgaggtagaa

SEQ ID NO: 23 - DNA, 21 bp, synthetic construct
  source 1..21: mol_type = other DNA
acctgccgacctgatgaattc

SEQ ID NO: 24 - DNA, 21 bp, synthetic construct
  source 1..21: mol_type = other DNA
gcagtcatgttcacggtcaca